(19) United States
(12) Patent Application Publication
(10) Pub. No.: US 2004/0220603 A1
(43) Pub. Date: Nov. 4, 2004

(54) Lancet

(76) Inventors: Wlodzimierz Rutynowski, Warsaw (PL); Wojciech Wyszogrodzki, Warsaw (PL)

Correspondence Address: Michael D Bednarek, Shaw Pittman, 1650 Tysons Boulevard, MCLean, VA, 22102, US

(21) Appl. No.: 10/484,449

(22) PCT Filed: Aug. 9, 2002
(86) PCT No.: PCT/PL02/00060

(30) Foreign Application Priority Data
Aug. 13, 2001 (PL) P349141

(51) Int. Cl. A61B 17/32

(52) U.S. Cl. 606/181

(57) ABSTRACT

A lancet comprises a handle (1) made of plastic material with a needle (2), where the tip (3) of the needle (2) is >covered by a sheath (4) moulded together with the handle (1). The handle (1) includes at least one hole expositing the needle (2). The part of the needle (2) positioned in at least one hole (5) of the handle (1) is crooked.

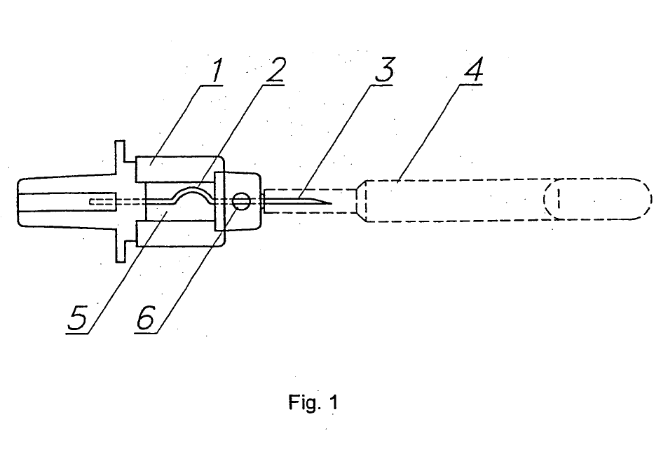

### BACKGROUND OF THE INVENTION

1. Field of the Invention

This invention relates to a lancet, in particular for a device for puncturing patien's skin in order to collect blood sample for the diagnostics purposes.

2. Description of the Related Art

The U.S. Pat. Nos. 4,379,456 and 5,554,166 disclose a device for puncturing comprises an elongated, generally cylindrical, housing having a chamber for a lancet holder. A lancet comprises a handle made of plastic material and a needle made of a solid stainless steel. The handle is moulded about the needle so that the tip of the needle extents outwardly beyond the upper end of the handle. Moreover, the lancet includes an integrally moulded needle sheath covering the pointed needle tip. The connection between the sheath and the handle is fragile so that twisting or rotating the sheath relative to the handle exposes the needle tip for puncturing patien's skin.

Further, the U.S. Pat. No. 5,100,427 discloses another construction of the lancet in the blood sampling device. The lancet has a moulded plastics body resembling the spool of a spool valve, with several annular ribs and a reduced diameter tail. It coaxially encases a needle whose tip is initially shielded and maintained in a sterile condition by a sheath moulded with the body but capable of being broken away. Between some of the ribs there are radial holes expositing the needle. They are formed as an incidental part of the manufacturing process.

### SUMMARY OF THE INVENTION

According to the present invention, a lancet comprises a handle made of plastic material with a needle, the tip of which being covered by a sheath moulded together with the handle, the handle includes at least one hole expositing the needle, where the part of the needle positioned in at least one hole of the handle is crooked.

An advantage of the solution according to the present invention is a fact that the needle is secured by pulling it from the handle of the lancet during breaking the sheath away.

### BRIEF DESCRIPTION OF THE DRAWINGS

The accompanying drawing, which is incorporated in, and form a part of the specification, illustrate an embodiment of the present invention and, together with the description, serve to explain the principles of the invention.

### DETAILED DESCRIPTION OF THE INVENTION

Reference will now be made in detail to the preferred embodiment of the invention, an example of which is illustrated in the accompanying drawing.

The lancet is settled in the device for puncturing patien's skin in order to collect blood sample for the diagnostics purposes. The lancet comprises a handle (1) made of plastic material with a needle (2) made of a solid stainless steel. The tip (3) of the needle (2) is covered by a sheath (4) moulded together with the handle (1) during manufacturing process. The connection between the sheath (4) and the handle (1) is fragile. This connection enables breaking the sheath (4) away the handle (1) of the lancet and uncovering the tip (3) of the needle (2) for puncturing patien's skin.

The handle (1) of the lancet has two holes (5, 6) expositing the needle (2). The part of the needle (2) positioned in the first hole (5) of the handle (1) is crooked.

We claim:

1. A lancet, in particular for a device for puncturing patien's skin in order to collect blood sample for the diagnostics purposes, comprising a handle (1) made of plastic material with a needle (2), the tip (3) of which being covered by a sheath (4) moulded together with the handle (1), the handle (1) includes at least one hole (5) expositing the needle (2), characterised in that the part of the needle (2) positioned in at least one hole (5) of the handle (1) is crooked.
